(19) United States
(12) Patent Application Publication
(10) Pub. No.: US 2016/0100544 A1
(43) Pub. Date: Apr. 14, 2016

(54) RESISTANCE AGAINST HYALOPERONOSPORA PARASITICA IN DIPLOTAXIS TENUIFOLIA

(71) Applicant: RIJK ZWAAN ZAADTEELT EN ZAADHANDEL B.V., De Lier (NL)

(72) Inventor: Franciscus Hermanus CARREE, De Lier (NL)

(21) Appl. No.: 14/974,186

(22) Filed: Dec. 18, 2015

Related U.S. Application Data
(63) Continuation-in-part of application No. PCT/EP2014/063151, filed on Jun. 23, 2014.

(30) Foreign Application Priority Data
Jun. 21, 2013 (EP) 13173181.2

(51) Int. Cl. A01H 5/10

(52) U.S. Cl. CPC A01H 5/10; A23L 1/2123; A23V 2002/00

(57) ABSTRACT

The invention relates to a rucola plant of the species Diplotaxis tenuifolia which may comprise genetic determinant B and optionally A that leads to expression of resistance against at least Hyaloperonospora parasitica isolates Pp0615 and Pp1248, which genetic determinant B is obtainable from a Diplotaxis tenuifolia plant which may comprise said genetic determinant, representative seed of which was deposited with the NCIMB under deposit number NCIMB 42151 and which genetic determinant A is obtainable from a Diplotaxis tenuifolia plant which may comprise said genetic determinant, representative seed of which was deposited with the NCIMB under deposit number NCIMB 41811. The invention further provides seeds that can grow into a plant that is resistant to at least Hyaloperonospora parasitica isolates Pp0615 and Pp1248, to progeny of the rucola plant, to propagation material suitable for producing the plant, to seed and to parts of the plant and food products which may comprise the plant parts.

### RELATED APPLICATIONS AND INCORPORATION BY REFERENCE

This application is a continuation-in-part application of international patent application Serial No. PCT/EP2014/063151 filed 23 Jun. 2014, which published as PCT Publication No. WO 2014/202781 on 24 Dec. 2014, which claims benefit of European patent application Serial No. 13173181.2 filed 21 Jun. 2013.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a Diplotaxis tenuifolia plant which may comprise a genetic determinant that leads to expression of resistance against Hyaloperonospora parasitica. The invention further relates to a source of resistance to Hyaloperonospora parasitica for use in breeding. The invention also relates to the seeds and progeny of such plants and to propagation material for obtaining such plants. Furthermore the invention relates to the use of the plants, seeds and propagation material that may comprise the genetic determinant as germplasm in a breeding program.

### BACKGROUND OF THE INVENTION

Diplotaxis tenuifolia is an edible plant species and member of the Brassicaceae, the mustard plant family, known for numerous other edible plants such as Sinapis alba (mustard), Brassica oleracea (e.g. broccoli, cabbage, cauliflower), Brassica rapa (e.g. turnip, Chinese cabbage), Brassica napus (e.g. rapeseed), Raphanus sativus (common radish), Armorica rusticana (horseradish) and many others. Other names for Diplotaxis tenuifolia include rucola, wild rocket, or just rocket. The plant is often confused with another Brassicaceae member, Eruca sativa, which looks very similar, is used for the same purposes, and is also called rucola, salad rocket, or arugula. E. sativa, however, is a different species that cannot be crossed with D. tenuifolia.

D. tenuifolia is a diploid and perennial species, native to Europe and Western Asia. It can be found throughout much of the temperate world where it has been naturalized. It is an erect mustard-like plant with branching stems that may exceed half a meter in height. It grows in clumps on the ground in a variety of habitats and is a common weed of roadsides and disturbed areas. It has long leaves which may be lobed or not. The foliage is aromatic when crushed. Atop the branches of the stem are bright yellow flowers with four rounded petals each about a centimeter long. The fruit is a straight, flat silique up to five centimeters long.

All over the world Diplotaxis tenuifolia is grown as a salad vegetable. Especially in Italy, D. tenuifolia is an important crop. In other countries this crop has the potential to become more important over the coming years. The use of the young leaves of these plants is traditional in the Mediterranean cuisine. Because of the popular taste, the low amount of calories, and decorative effect, rocket salad is now very often appearing in many other dishes.

In Europe as well as in many other areas the production of rucola is being hindered by the infection of the plants by downy mildew (Hyaloperonospora parasitica, previously called Peronospora parasitica). Downy mildew is a polycyclic disease caused by different species of the oomycete Hyaloperonospora. The obligate parasite from the genus Hyaloperonospora is living on Brassica plants and related cruciferous crops. The losses are more severe at the seedling stage than on mature healthy plants. Young seedlings can die as a result of the infection. Cool and moist conditions are favorable for the disease development. The disease causes quantitative and qualitative losses of the crops. Although all (aerial) parts of the plant can be infected, the symptoms primarily appear on the leaves. Symptoms of the disease such as discolored lesions and necrotic spots with brown edges can destroy the quality of the leaves. Also the quantity of the harvest is reduced due to the downy mildew infection.

None of the varieties currently available on the market are resistant to downy mildew. Gene bank accessions were tested during internal research but no resistance was identified.

Citation or identification of any document in this application is not an admission that such document is available as prior art to the present invention.

### SUMMARY OF THE INVENTION

It is thus an object of the present invention to provide a Diplotaxis tenuifolia plant that is resistant to Hyaloperonospora parasitica.

During the research that led to the present invention two different Hyaloperonospora parasitica isolates, Pp0615 and Pp1248, were identified and a differential reference set of Diplotaxis tenuifolia cultivars was set up to allow identification of these Hyaloperonospora parasitica isolates. Downy mildew isolate Pp1248 was identified only recently. This new isolate may spread during the next growing seasons and cause economic damage to the worldwide rucola industry. Hyaloperonospora parasitica isolates Pp0615 and Pp1248 may be obtained upon request from the applicant, Rijk Zwaan. As for oomycetes like Hyaloperonospora parasitica it is known that they continuously develop the ability to break resistances present in their host plant, new resistance loci are very valuable assets. During the research that led to the present invention new Diplotaxis tenuifolia plants were created that exhibit resistance against at least the newly identified Hyaloperonospora parasitica isolates Pp0615 and Pp1248. Several sources were used to create these plants, all of them showing susceptibility to downy mildew. Surprisingly, rucola material with a high level of resistance against Hyaloperonospora parasitica, the downy mildew that infects rucola, was developed on the basis of these susceptible sources.

The present invention thus provides Diplotaxis tenuifolia plants which may comprise genetic determinant B which in homozygous state leads to the expression of resistance against at least Hyaloperonospora parasitica isolates Pp0615 and Pp1248. The resistance is preferably a complete resistance. Determinant B is thus preferably present in homozygous state. Genetic determinant B is as present in the genome of, or obtainable from, plants grown from seeds of which a representative sample was deposited at the NCIMB under deposit number NCIMB 42151. This genetic determinant may optionally be combined with genetic determinant A which also leads to the resistance to downy mildew of the invention and is as present in the genome of, or obtainable from, plants grown from seeds of which a representative sample was deposited at the NCIMB under deposit number NCIMB 41811.

In one embodiment, the rucola plant of the invention carrying genetic determinant B, preferably in homozygous state, is obtainable by crossing a first rucola plant with a second rucola plant, wherein one or both of the said plants is grown from seed which may comprise genetic determinant B of which a representative sample was deposited with the NCIMB under deposit number NCIMB 42151, or a progeny plant thereof, and selecting in the F2 for a plant that shows resistance to at least Hyaloperonospora parasitica isolates Pp0615 and Pp1248. In addition genetic determinant A may be introgressed from a rucola plant representative seed of which has been deposited with the NCIMB under deposit number NCIMB 41811.

Selection for a plant that shows resistance to downy mildew is preferably done in the F2 and/or in any subsequent generation in which the resistance trait is visible or segregates.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSITS

Seeds of Diplotaxis tenuifolia 13.26717 that comprise the genetic determinant B of the invention which in a homozygous state leads to resistance to at least Hyaloperonospora parasitica isolates Pp0615 and Pp1248, were deposited with NCIMB Ltd, Ferguson Building, Craibstone 5 Estate, Bucksburn, Aberdeen AB21 9YA, UK on 14 Jun. 2013 under deposit accession number NCIMB 42151. Seeds of Diplotaxis tenuifolia 11.87212 that comprise genetic determinant A which leads to resistance to at least Hyaloperonospora parasitica isolates Pp0615 and Pp1248, were deposited with NCIMB Ltd, Ferguson Building, Craibstone 5 Estate, Bucksburn, Aberdeen AB21 9YA, UK on 16 Feb. 2011 under deposit accession number NCIMB 41811.

The Deposits with NCIMB Ltd, Ferguson Building, Craibstone 5 Estate, Bucksburn, Aberdeen AB21 9YA, UK, under deposit accession numbers NCIMB 41811 and NCIMB 42151 were made pursuant to the terms of the Budapest Treaty. Upon issuance of a patent, all restrictions upon the deposit will be removed, and the deposit is intended to meet the requirements of 37 CFR §§1.801-1.809. The deposit will be irrevocably and without restriction or condition released to the public upon the issuance of a patent. The deposit will be maintained in the depository for a period of 30 years, or 5 years after the last request, or for the effective life of the patent, whichever is longer, and will be replaced if necessary during that period.

### DETAILED DESCRIPTION OF THE INVENTION

The resistance to at least downy mildew (Hyaloperonospora parasitica) isolates Pp0615 and Pp1248 is a high level resistance, preferably a complete resistance. A high level of resistance is expressed by plants showing a reduced level of infection with the formation of necrotic lesions but only little or no sporulation, when these plants are exposed to a normal dose of the pathogen under common favorable conditions for pathogen growth. Complete resistance is expressed by symptomless plants, when these plants are exposed to a normal dose of the pathogen under said common favorable conditions. When the plants of the invention are tested for downy mildew in a bio-assay using spores of either isolate Pp0615 or Pp1248, they are completely free of downy mildew symptoms. Several bio-assays were carried out to confirm the complete level of resistance. The complete resistance was also confirmed in a field trial in which none of the plants of the invention showed downy mildew symptoms while other rucola plants showed symptoms of infection. Seeds from rucola plants that have complete resistance against at least downy mildew isolates Pp0615 and Pp1248, were deposited with the NCIMB under deposit number NCIMB 41811 and NCIMB 42151.

It was found that the resistance to downy mildew of the invention of plants which may comprise the genetic determinant B is inherited in a semi-dominant or incomplete dominant way. In cases of incomplete dominance or semi-dominance, the phenotype of the heterozygote is intermediate between those of the parent homozygotes. In this case this means the phenotype of the heterozygotes is intermediate between expressing a high resistance, in particular a complete resistance, and a susceptible reaction. This type of inheritance may also be indicated as intermediate inheritance. Genetic determinant B must be present in a homozygous state to result in the phenotypic trait of the invention.

Preferably the resistance of plants which may comprise the genetic determinant B against at least Hyaloperonospora parasitica isolates Pp0615 and Pp1248, is transmitted by a semi-dominant or incomplete dominant inheritance, in particular when the plant that may comprise the genetic determinant is used as the female parent in a cross. The resulting F1 from a cross between a resistant Diplotaxis tenuifolia plant of the invention which may comprise genetic determinant B and a susceptible D. tenuifolia plant has an intermediate resistance to Hyaloperonospora parasitica isolates Pp0615 and Pp1248. Intermediate resistance in this context means the plants will under a low disease pressure, i.e. a low dose of the pathogen, show no disease symptoms or symptoms limited to the formation of necrotic lesions with only little or no sporulation, while under a normal to high disease pressure such plants will show both the formation of necrotic lesions and sporulation. In a mildly infected field, plants which may comprise the second genetic determinant will thus show no disease symptoms or symptoms limited to the formation of necrotic lesions with only little or no sporulation. In a bio-assay such as the one described below and in example 2 such plants will in many cases show disease symptoms ranging from only the formation of necrotic lesions to sporulation. In the F2 the resistance segregates and resistant plants can be selected. Selection in the F2 for plants that have a high level resistance, in particular a complete resistance, favors the selection of plants that may comprise genetic determinant B in a homozygous state. To verify such selected F2 plants indeed comprise genetic determinant B in a homozygous state the resistance level of the corresponding F3 populations may be examined.

It was found that the resistance to downy mildew of the invention of plants which may comprise genetic determinant A is inherited in a dominant way. Genetic determinant A may be present in a homozygous or heterozygous state to result in the phenotypic trait of the invention.

Preferably the resistance of plants which may comprise genetic determinant A against at least Hyaloperonospora parasitica isolates Pp0615 and Pp1248, is transmitted by dominant inheritance, in particular when the plant that may comprise the genetic determinant is used as the female parent in a cross. The resulting F1 from a cross between a resistant Diplotaxis tenuifolia plant of the invention and a susceptible D. tenuifolia plant has a high resistance, in particular a complete resistance. In the F2 the resistance segregates and resistant plants can be selected.

Selection of plants that have a high resistance, in particular a complete resistance, can be done by performing a bio-assay under controlled conditions that favour the development of downy mildew on the rucola plants.

The invention relates to a Diplotaxis tenuifolia plant which may comprise genetic determinant B which inherits as a single semi-dominant gene and in a homozygous state leads to a high, in particular a complete resistance against Hyaloperonospora parasitica, in particular resistance against at least Hyaloperonospora parasitica isolates Pp0615 and Pp1248, as demonstrated by an absence of downy mildew symptoms, which genetic determinant is as present in a Diplotaxis tenuifolia plant which may comprise said genetic determinant, representative seed of which was deposited with the NCIMB under deposit number NCIMB 42151, and optionally genetic determinant A which inherits as a single dominant gene and leads to a high, in particular a complete resistance against Hyaloperonospora parasitica, in particular resistance against at least Hyaloperonospora parasitica isolates Pp0615 and Pp1248, as demonstrated by an absence of downy mildew symptoms, which genetic determinant is as present in a Diplotaxis tenuifolia plant which may comprise said genetic determinant, representative seed of which was deposited with the NCIMB under deposit number NCIMB 41811. In order to obtain a complete resistance determinant B should be present in homozygous state. Determinant A also shows complete resistance in heterozygous state. Plants of the invention showing complete resistance can be homozygous for B and optionally heterozygous for A or homozygous for A. Plants that have determinant A either homozygously or heterozygously and determinant B in either homozygous or heterozygous state are completely resistant. The genetic determinants and the resistance phenotype caused by the determinants are thus obtainable from plants grown from the deposited seeds.

The invention preferably relates to a Diplotaxis tenuifolia plant which may comprise genetic determinant B homozygously, and optionally genetic determinant A, as demonstrated in a bio-assay for Hyaloperonospora parasitica, wherein a relevant number of plants, such as 30, is grown for about 2 weeks at a temperature regime of 14/12° C. day/night before being inoculated with spores of Hyaloperonospora parasitica isolates Pp0615 or Pp1248, after which the plants are scored for infection at 10 and 17 days after inoculation, and wherein plants of the invention are characterized by an absence of downy mildew symptoms. In such a Hyaloperonospora parasitica bioassay suitable controls, in particular rucola reference cultivars Soria (Gautier semences, France) and/or Reset (Ortis quality seeds, Italy), are included.

A skilled rucola breeder knows how to obtain a resistant D. tenuifolia plant which may comprise both genetic determinant B in a homozygous state and determinant A.

For obtaining a resistant hybrid of rucola which may comprise genetic determinant A, the line used as the female preferably may comprise genetic determinant A of the invention that leads to resistance to downy mildew. For obtaining a resistant hybrid of rucola which may comprise genetic determinant B, both parental lines may comprise genetic determinant B of the invention that leads to resistance to downy mildew.

In one embodiment, a plant which may comprise the genetic determinant B that leads to resistance to at least downy mildew isolates Pp0615 and Pp1248, is obtainable by introgression from a rucola plant, representative seed of which has been deposited with the NCIMB under deposit number NCIMB 42151. In addition genetic determinant A may be introgressed from a rucola plant representative seed of which has been deposited with the NCIMB under deposit number NCIMB 41811.

“Introgression” as used herein is intended to mean introduction of a genetic determinant that confers a trait into a plant not carrying the genetic determinant by means of crossing and selection in the first generation in which the trait becomes visible. Selection can alternatively start in any subsequent generation in which the trait is visible or segregates.

The invention also relates to tissue of a plant as claimed. The tissue can be undifferentiated tissue or already differentiated tissue. Undifferentiated tissues are for example stem tips, anthers, petals, pollen and can be used in micropropagation to obtain new plantlets that are grown into new plants of the invention.

The invention furthermore relates to a cell of a rucola plant as claimed, which cell may comprise genetic determinant B, preferably homozygously, which confers the downy mildew resistance of the invention, wherein said genetic determinant B is as present in the genome of a rucola plant grown from seeds of which a representative sample was deposited with the NCIMB under deposit number NCIMB 42151. The cell optionally may further comprise determinant A. Said rucola plant is obtainable by crossing a first rucola plant with a second rucola plant, in particular a rucola plant grown from seed as deposited under deposit number NCIMB 42151, and selecting for a rucola plant that has the resistance trait of the invention. The said cell thus may comprise the genetic information which is substantially identical, preferably completely identical to the genetic information encoding the said resistance trait of the rucola plant grown from seeds of which a representative sample was deposited under deposit number NCIMB 42151, more in particular genetic determinant B described herein, and optionally introgressing A. Preferably, the cell of the invention is a part of a plant or plant part, but the cell may also be in isolated form.

In one embodiment, the invention relates to the use of seeds with NCIMB accession number NCIMB 42151 for transferring genetic determinant B of the invention, which confers the resistance trait of the invention, into another rucola plant.

In a further embodiment, the invention relates to the use of seeds with NCIMB accession number NCIMB 41811 for transferring genetic determinant A into a rucola plant which may comprise determinant B.

In another embodiment, the invention relates to the use of a rucola plant, which plant carries genetic determinant B of the invention which confers the resistance trait of the invention, as present in and obtainable from a rucola plant, in particular a rucola plant grown from seed which may comprise genetic determinant B as deposited under deposit number NCIMB 42151, optionally in combination with determinant A, as a crop. The invention also relates to the use of a rucola plant, which carries genetic determinant B of the invention which confers the resistance trait of the invention, as present in and obtainable from a rucola plant, in particular a rucola plant grown from seed which may comprise genetic determinant B as deposited under deposit number NCIMB 42151, optionally in combination with determinant A, as a source of seed.

In yet another embodiment, the invention relates to the use of a rucola plant, which carries genetic determinant B which confers the resistance trait of the invention, as present in and obtainable from a rucola plant, in particular a rucola plant grown from seed which may comprise genetic determinant B as deposited under deposit number NCIMB 42151, optionally in combination with determinant A, as a source of propagating material.

Further, the invention relates to the use of a rucola plant, which carries genetic determinant B which confers the resistance trait of the invention, as present in and obtainable from a rucola plant, in particular a rucola plant grown from seed which may comprise genetic determinant B as deposited under deposit number NCIMB 42151, optionally in combination with determinant A, for consumption.

In another embodiment, the invention relates to the use of a rucola plant, which carries genetic determinant B which confers the resistance trait of the invention as present in seed which may comprise genetic determinant B as deposited under deposit number NCIMB 42151, optionally in combination with determinant A, for conferring the genetic determinant that leads to the resistance trait of the invention to a rucola plant.

In yet another embodiment, the invention relates to the use of a rucola plant, as a recipient of genetic determinant B, optionally in a homozygous state, as present in seed as deposited under deposit number NCIMB 42151 and optionally of determinant A as present in seed as deposited under deposit number NCIMB 41811.

The invention also relates to plant parts, in particular leaves and stems, which are produced by a plant of the invention. The invention further relates to a food product, which may comprise the leaves and/or stems of a rucola plant of the invention, or parts thereof. A preferred food product which may comprise parts of the rucola plant of the invention is a salad, wherein the rucola leaves may optionally be mixed with other leaves of for example lettuce, endive, chicory, beet, Swiss chard, spinach, etc.

The food product or harvested part, may have undergone one or more processing steps. Such a processing step might comprise but is not limited to any one of the following treatments or combinations thereof: cutting, washing, cooking, steaming, baking, frying, pasteurizing, freezing, grinding, extracting oil, pickling, or fermenting. The processed form that is obtained is also part of this invention.

The processed rucola may also be included in another food product, such as a sauce, pesto, pie, soup or a dried or fresh pasta product, such as ravioli, tortellini, cannelloni etc. Such food product will usually be pre-packed and is intended for sale in a supermarket. The invention thus also relates to the use of rucola plants of the invention or parts thereof in the preparation of food products, in particular salads, pies, soups and pastas.

The invention according to a further aspect thereof relates to seeds of a rucola plant, which may comprise genetic determinant B, preferably in a homozygous state, which leads to resistance to at least Hyaloperonospora parasitica isolates Pp0615 and Pp1248. In addition, genetic determinant A may also be introgressed into said rucola plant. Although the seeds do not show the trait of the rucola plant of the invention, they harbour the genetic information that when a plant is grown from the seeds makes this plant a plant of the invention.

The invention also relates to seed that is capable of growing into a rucola plant that is resistant to at least Hyaloperonospora parasitica isolates Pp0615 and Pp1248, wherein the seed may comprise genetic determinant B, preferably in a homozygous state, and optionally determinant A, which genetic determinant or combination of genetic determinants leads to the downy mildew resistance of the invention.

The invention furthermore relates to hybrid seed and to a method for producing hybrid seed which may comprise crossing a first parent plant with a second parent plant and harvesting the resultant hybrid seed, wherein said first parent plant and/or said second parent plant is the plant as claimed.

The invention also relates to inbreds and doubled haploids of rucola plants of the invention.

The invention also relates to progeny of the plants, cells, tissues and seeds of the invention. Such progeny can in itself be plants, cells, tissues or seeds. Such progeny may be produced by sexual and vegetative reproduction of a plant of the invention or a progeny plant thereof. The invention relates to progeny derived from a rucola plant of the invention, or from rucola seed that harbours the trait of the invention, which progeny is resistant to at least Hyaloperonospora parasitica isolates Pp0615 and Pp1248. In addition to this, the plant may be modified in one or more other characteristics. Such additional modifications are for example effected by crossing and selecting, mutagenesis or by transformation with a transgene.

As used herein the word “progeny” is intended to mean the offspring or the first and all further descendants from a cross with a plant of the invention that, preferably in homozygous state, may comprise genetic determinant B that in a homozygous state leads to a high resistance, in particular a complete resistance against at least Hyaloperonospora parasitica isolates Pp0615 and Pp1248. Progeny of the invention may comprise descendants of any cross with a plant of the invention that carries the trait that leads to resistance against downy mildew. Such progeny is for example obtainable by crossing a first rucola plant with a second rucola plant, wherein one or both of the plants were grown from seeds which may comprise genetic determinant B, representative seed of which was deposited with the NCIMB under deposit number NCIMB 42151, but may also be the progeny of any other rucola plant carrying genetic determinant B of the invention as present in NCIMB 42151. In addition genetic determinant A, as present in NCIMB 41811, may also be comprised therein. “Progeny” also encompasses plants that carry the trait of the invention and are obtained from other plants or progeny of plants of the invention by vegetative propagation or multiplication.

Propagation material derived from a rucola plant of the invention or from rucola seeds from a rucola plant of the invention, is also included in the present invention, wherein the propagation material may comprise genetic determinant B, preferably in a homozygous state and optionally A that cause the downy mildew resistance of the invention.

The invention also refers to propagation material capable of growing into a rucola plant of the invention.

The said propagation material, derived from the rucola plant of the invention as well as propagation material capable of growing into a plant of the invention is for example selected from the group consisting of callus, microspores, pollen, ovaries, ovules, embryos, embryo sacs, egg cells, cuttings, roots, stems, cells, protoplasts, leaves, cotyledons, hypocotyls, meristematic cells, roots, root tips, microspores, anthers, flowers, seeds and stems or parts or tissue culture thereof.

The invention further relates to seed of the claimed plant and to parts of the plant that are suitable for sexual reproduction. Such parts are for example selected from the group consisting of microspores, pollen, ovaries, ovules, embryo sacs and egg cells.

In addition, the invention also relates to parts of the rucola plant of the invention that are suitable for vegetative reproduction, for example tissue culture, cuttings, roots, stems, cells, and protoplasts.

According to a further aspect thereof the invention provides a tissue culture of the claimed plant. The tissue culture suitably may comprise regenerable cells. Such tissue culture can be derived from leaves, pollen, embryos, cotyledons, hypocotyls, meristematic cells, roots, root tips, anthers, flowers, seeds and stems.

The invention further relates to rucola plants of the invention that carry, preferably in homozygous state, genetic determinant B and optionally A which lead to resistance to at least Hyaloperonospora parasitica isolates Pp0615 and Pp1248, and having acquired said one or more determinants by introduction of the genetic information that is responsible for the trait from a suitable source, either by conventional breeding, or genetic modification, in particular by cisgenesis or transgenesis. Cisgenesis is genetic modification of plants with a natural gene, coding for an (agricultural) trait, from the crop plant itself or from a sexually compatible donor plant. Transgenesis is genetic modification of a plant with a gene from a non-crossable species or a synthetic gene.

The invention also relates to a method for production of a Diplotaxis tenuifolia plant which may comprise resistance to at least Hyaloperonospora parasitica isolates Pp0615 and Pp1248, which may comprise
a) crossing a plant which may comprise genetic determinant B that in a homozygous state leads to expression of resistance to least Hyaloperonospora parasitica isolates Pp0615 and Pp1248, with another plant;
b) selecting resistant plants;
c) optionally performing one or more rounds of selfing or crossing, and subsequently selecting, for a plant which may comprise resistance against downy mildew, in particular resistance against at least downy mildew isolates Pp0615 and Pp1248, wherein genetic determinant B is as found in and is obtainable from a Diplotaxis tenuifolia plant of which representative seed was deposited under number NCIMB 42151. Selecting of resistant plants may be done in the F2 or any further generation, but is preferably done in the F2. It is clear that the parent that provides the trait of the invention is not necessarily a plant grown directly from the deposited seeds. The parent can also be a progeny plant from the seed or a progeny plant from seeds that are identified to have the trait of the invention by other means.

In one aspect, the invention relates to a method for production of a Diplotaxis tenuifolia plant which may comprise resistance to at least Hyaloperonospora parasitica isolates Pp0615 and Pp1248, which may comprise:
a) crossing a plant which may comprise genetic determinant B that in a homozygous state leads to expression of Hyaloperonospora parasitica resistance to at least Hyaloperonospora parasitica isolates Pp0615 and Pp1248 with another plant;
b) optionally backcrossing plants expressing an intermediate resistance from the resulting F1 with the preferred parent;
c) selecting plants expressing an intermediate resistance in the next generation; and
d) selfing selected plants and in the next generation selecting plants which may comprise a high level of resistance, in particular a complete resistance against downy mildew; and
e) optionally performing one or more additional rounds of selfing or crossing, and subsequently selecting, for a plant which may comprise resistance against downy mildew,
   wherein genetic determinant B is as found in and is obtainable from a Diplotaxis tenuifolia plant of which representative seed was deposited under number NCIMB 42151.

The invention additionally provides a method of introducing a desired trait into a rucola plant which may comprise the resistance trait of the invention, which may comprise:
a) crossing a Diplotaxis tenuifolia plant which may comprise the resistance trait of the invention, representative seed of which was deposited with the NCIMB under deposit number NCIMB 42151, with a second rucola plant that may comprise a desired trait to produce F1 progeny;
b) selecting in the F1 progeny plants that express an intermediate resistance, and the desired trait;
c) crossing the selected F1 progeny with either parent, to produce backcross progeny;
d) selecting backcross progeny which may comprise the desired trait and the resistance trait of the invention; and
e) optionally repeating steps (c) and (d) one or more times in succession to produce selected fourth or higher backcross progeny that may comprise the desired trait and resistance against downy mildew. The invention includes a rucola plant produced by this method.

The plants obtained by the above methods may further comprise or acquire genetic determinant A.

In one embodiment selection for intermediately resistant plants is done in the F1. In another aspect, selection for resistant plants is started in the F2 of a cross or alternatively of a backcross. Selection of highly resistant plants, preferably completely resistant plants, can be done by performing a bio-assay under controlled conditions that favour the development of downy mildew on the rucola plants, for example a bio-assay such as the one described in example 2.

In one embodiment selection for resistant plants is started in the F3 or a later generation.

In one embodiment the plant which may comprise genetic determinant B and optionally A is a plant of an inbred line, a hybrid, a doubled haploid, or of a segregating population.

The invention further provides a method for the production of a Diplotaxis tenuifolia plant which may comprise resistance to at least Hyaloperonospora parasitica isolates Pp0615 and Pp1248, by using a doubled haploid generation technique to generate a doubled haploid line which may comprise the said resistance.

In one embodiment, the invention relates to a method for producing a hybrid Diplotaxis tenuifolia plant which may comprise crossing a first parent rucola plant with a second parent rucola plant and harvesting the resultant hybrid rucola seed, in which the first parent rucola plant and the second parent rucola plant may comprise the resistance against at least Hyaloperonospora parasitica isolates Pp0615 and Pp1248, as found in Diplotaxis tenuifolia plants of which representative seed was deposited under number NCIMB 42151 and optionally the first parent rucola plant and/or the second parent rucola plant may comprise the resistance against at least Hyaloperonospora parasitica isolates Pp0615 and Pp1248, as found in Diplotaxis tenuifolia plants of which representative seed was deposited under number NCIMB 41811.

The invention also relates to a method for the production of a rucola plant which may comprise resistance against at least Hyaloperonospora parasitica isolates Pp0615 and Pp1248, by using a seed that may comprise downy mildew resistance in its genome, for growing the said rucola plant. The seeds are suitably seeds of which a representative sample was deposited with the NCIMB under deposit number NCIMB 42151. The rucola plant may optionally further comprise genetic determinant A.

The invention also relates to a method for seed production which may comprise growing plants from seeds of which a representative sample was deposited with the NCIMB under deposit number NCIMB 42151, allowing the plants to produce seeds, and harvesting those seeds. The rucola seeds may optionally further comprise genetic determinant A. Production of the seeds is suitably done by crossing or selfing.

Further, the invention relates to a method for the production of a Diplotaxis tenuifolia plant which may comprise resistance against at least Hyaloperonospora parasitica isolates Pp0615 and Pp1248, as found in and obtainable from a Diplotaxis tenuifolia plant of which representative seed was deposited under number NCIMB 42151, by using tissue culture. The rucola plant may optionally further comprise genetic determinant A.

The invention furthermore relates to a method for the production of a Diplotaxis tenuifolia plant which may comprise the resistance against at least Hyaloperonospora parasitica isolates Pp0615 and Pp1248, by using vegetative reproduction.

In one embodiment, the invention relates to a method for the production of a Diplotaxis tenuifolia plant which may comprise the resistance against at least Hyaloperonospora parasitica isolates Pp0615 and Pp1248, by using a method for genetic modification to introgress the resistance into the rucola plant. Genetic modification may comprise transgenic modification or transgenesis, using a gene from a non-crossable species or a synthetic gene, and cisgenic modification or cisgenesis, using a natural gene, coding for an (agricultural) trait, from the crop plant itself or from a sexually compatible donor plant.

In one embodiment, the source from which the genetic information is acquired, in particular genetic determinant B and optionally A, is formed by a plant grown from the deposited seeds, or by sexual or vegetative descendants thereof.

The invention also relates to the germplasm of plants of the invention. The germplasm is constituted by all inherited characteristics of an organism and according to the invention encompasses at least the resistance trait of the invention. The germplasm can be used in a breeding program for the development of rucola plants that are resistant against downy mildew. The invention also relates to a breeding method for the development of Diplotaxis tenuifolia plants that are resistant against at least Hyaloperonospora parasitica isolates Pp0615 and Pp1248, wherein germplasm which may comprise resistance against at least Hyaloperonospora parasitica isolates Pp0615 and Pp1248, is used. Representative seed of said plant which may comprise genetic determinant B was deposited with the NCIMB under deposit number NCIMB 42151. The germplasm may optionally further comprise genetic determinant A.

In one embodiment the invention relates to a method for the production of a Diplotaxis tenuifolia plant which may comprise resistance against at least Hyaloperonospora parasitica isolates Pp0615 and Pp1248, wherein progeny or propagation material of a plant which may comprise genetic determinant B and optionally A conferring said resistance is used as a source to introgress the resistance into another rucola plant. Representative seed of said plant which may comprise genetic determinant B was deposited with the NCIMB under deposit number NCIMB 42151.

The invention provides preferably a rucola plant having the trait of resistance to at least Hyaloperonospora parasitica isolates Pp0615 and Pp1248, which plant is obtainable by any of the methods described herein and/or familiar to the skilled person.

Furthermore, the invention relates to a downy mildew resistance gene that in a homozygous state leads to a rucola plant having a high resistance, in particular a complete resistance against at least Hyaloperonospora parasitica isolates Pp0615 and Pp1248, and which resistance gene is as present in the genome of plants of which a representative sample was deposited with the NCIMB under deposit number NCIMB 42151. The skilled breeder knows how to use such plant as a source of the resistance gene for introgressing the resistance gene into a plant.

The invention also relates to the use of genetic determinant B and optionally in combination with determinant A that lead to a rucola plant having a high resistance, in particular a complete resistance against at least Hyaloperonospora parasitica isolates Pp0615 and Pp1248, for producing a plant which is resistant to downy mildew, in particular a rucola plant which is resistant to downy mildew, which genetic determinant B is as present in the genome of plants of which a representative sample was deposited under deposit number NCIMB 42151 and which genetic determinant A is as present in the genome of plants of which a representative sample was deposited under deposit number NCIMB 41811.

According to another aspect thereof the invention relates to a non-naturally occurring plant having a high resistance, in particular a complete resistance against at least Hyaloperonospora parasitica isolates Pp0615 and Pp1248, and which resistance is the result of the presence in the genome of the plant of genetic determinant B which is as present in the genome of plants of which a representative sample was deposited under deposit accession number 42151, and optionally the presence in the genome of the plant of genetic determinant A which is as present in the genome of plants of which a representative sample was deposited under deposit accession number NCIMB 41811. The non-naturally occurring plant is in particular a mutant plant.

The term ‘genetic determinant’ as used herein

encompasses one or more genes or alleles. These terms are used interchangeably.

The ‘genetic trait’ is the trait or characteristic that is conferred by the genetic determinant. The genetic trait can be identified phenotypically, for example by performing a bio-assay. However, also plant stages for which no phenotypic assay can be performed do carry the genetic information that leads to the genetic trait. ‘Trait’ or ‘phenotypic trait’ can be used instead of ‘genetic trait’.

In the absence of molecular markers, equivalence of genetic determinants can be determined by an allelism test. To perform an allelism test, material that is homozygous for the known determinant is crossed with material that is homozygous for the phenotypic trait to be tested. When no segregation for the trait to be observed is present in the F2 of the cross, the genetic determinants resulting in the phenotypic trait have been proven to be the same.

When more than one gene is responsible for a certain trait, and an allelism test is done to determine equivalence, the skilled person doing the test has to ascertain that all relevant genes are present homozygously for the test to work properly.

To determine the presence of a resistance, a bio-assay can be carried out. Bio-assays can usually be performed in several ways, as known by persons skilled in the art. One way of performing the bio-assay for determining resistance of rucola to downy mildew is described above and in Example 2.

The resistance of the invention as mentioned herein is a high level resistance and preferably a complete resistance to at least downy mildew isolates Pp0615 and Pp1248. A high level of resistance is expressed by plants showing a reduced level of infection with the formation of necrotic lesions but only little or no sporulation, when these plants are exposed to a normal dose of the pathogen under common favorable conditions for growth of the pathogen. Complete resistance is expressed by symptomless plants, when these plants are exposed to a normal dose of the pathogen under said common favorable conditions. Plants are, for example, exposed to a normal dose of the pathogen under said common favorable conditions when the bio-assay as described above, or in Example 2, is carried out, and/or in a downy mildew infected field.

As used throughout this application, rucola encompasses only Diplotaxis tenuifolia plants, and does not include Eruca sativa plants. The terms rucola and Diplotaxis tenuifolia can be used interchangeably.

As used throughout this application, downy mildew resistance encompasses resistance against at least Hyaloperonospora parasitica isolates Pp0615 and Pp1248. Hyaloperonospora parasitica was previously called, and is in some instances still called, Peronospora parasitica. The terms downy mildew, Hyaloperonospora parasitica and Peronospora parasitica can be used interchangeably.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Creation of Diplotaxis tenuifolia Plants of the Invention

To obtain resistance against downy mildew, a variation of germplasm from the breeding program was screened through the performance of bio-assays. No resistant germplasm could be identified.

The performance of bio-assays on offspring of combinations of germplasm that were highly susceptible however resulted in the identification of some plants with resistance to at least Hyaloperonospora parasitica isolates Pp0615 and Pp1248. Continued inbreeding and screening for the relevant trait led to the creation of completely resistant D. tenuifolia plants from two different combinations of germplasm. In both cases the complete resistance was confirmed to be stable by the performance of a bio-assay screen on three subsequent generations, and no segregation for the trait of the invention was observed. An allelism test was performed which demonstrated the completely resistant D. tenuifolia plants from the two different combinations of germplasm were not allelic, but in fact comprised two different genetic determinants, A and B. After several generations the seeds were harvested and deposited under accession number NCIMB 41811 and NCIMB 42151. The seeds deposited under accession number NCIMB 41811 comprise genetic determinant A in a homozygous state, while the seeds deposited under accession number NCIMB 42151 comprise genetic determinant B in a homozygous state.

### Example 2

### Bio-Assay for Hyaloperonospora parasitica

To test whether a plant is resistant to at least Hyaloperonospora parasitica isolates Pp0615 and Pp1248, a bio-assay is performed. The bio-assay can be done in the following way.

Seeds are sown, along with those of wild rocket reference cultivars Reset (Ortis quality seeds, Italy) and Soria (Gautier semences, France), for example in 4 cm peat potting blocks. The plants are grown for 2 weeks at a temperature regime of 14/12° C. day/night. A relevant number of plants per line are evaluated, e.g. 30 plants, so that segregation can be observed if it would be present. After 2 weeks the young plants are inoculated with spores of either Hyaloperonospora parasitica isolate Pp0615 or Hyaloperonospora parasitica Pp1248. The plants are scored for infection 10 days after inoculation, and again at 17 days after inoculation. Plants are scored as resistant or susceptible based on symptoms of necrosis and signs of pathogen sporulation on the cotyledons and true leaves. Plants exhibiting more than a little sporulation are considered susceptible. Plants without any symptoms of downy mildew are completely resistant.

Table 1 shows the differential set of Hyaloperonospora parasitica isolates and the resistance of two wild rocket reference cultivars to each of the Hyaloperonospora parasitica isolates. A susceptible reaction is scored as “+” (indicating a successful infection by Hyaloperonospora parasitica, with the both necrosis and sporulation occurring), a susceptible reaction showing a reduced level of infection with the formation of necrotic lesions but only little or no sporulation is indicated as “(+)”, and resistance is depicted as “−”. A susceptible reaction showing a reduced level of infection may also be called an intermediate resistance or a tolerance to downy mildew.

TABLE 1
Differential set of downy mildew isolates and the resistance of
various Diplotaxis tenuifolia cultivars to each of these
pathogenic isolates.

| | | | Resistance of | Resistance of |
|---|---|---|---|---|
| | | | the invention | the invention |
| | | | which comprise | which comprise |
| | | | the genetic | the genetic |
| | | | determinant A | determinant B |
| | | | homozygously, | homozygously, |
| | | | deposit line | deposit line |
| Isolate | Soria | Reset | NCIMB 41811 | NCIMB 42151 |
| Pp0615 | + | + | − | − |
| Pp1248 | (+) | (+) | − | − |

### Example 3

### Transfer of the Trait of the Invention to Other Rucola Plants

Resistant rucola plants that were created as described in Example 1 were used to develop other resistant rucola plants. A completely resistant D. tenuifolia plant which may comprise genetic determinant A was crossed with a susceptible D. tenuifolia plant. Unexpectedly, the resulting heterozygous F1 was completely resistant to at least Hyaloperonospora parasitica isolates Pp0615 and Pp1248. This result indicates dominant inheritance of the trait of the invention in plants which may comprise the genetic determinant A.

Subsequently these completely resistant F1 plants were selfed and F2 plants were obtained. In the F2 74% completely resistant plants could be identified, and 26% of the plants showed symptoms of downy mildew after inoculation (Table 2). This further confirms the dominant inheritance of genetic determinant A of the invention. The F2 segregation ratio also establishes that the resistance to at least Hyaloperonospora parasitica isolates Pp0615 and Pp1248, is conferred by a monogenetic dominant determinant A.

TABLE 2
Segregation of the trait in the F2 of a completely resistant D. tenuifolia
plant which comprise genetic determinant A and a susceptible
D. tenuifolia plant.

| | | | | # F2 | |
|---|---|---|---|---|---|
| | # observed | | | plants | |
| | F2 | # resistant | % | with | % |
| parents | populations | F2 plants | resistant | symptoms | susceptible |
| (R × S)F2 | 17 | 252 | 75% | 86 | 25% |
| (S × R)F2 | 26 | 317 | 73% | 116 | 27% |
| Total | 43 | 569 | 74% | 202 | 26% |

A completely resistant D. tenuifolia plant which may comprise genetic determinant B was crossed with a susceptible D. tenuifolia plant. Unexpectedly, the phenotype of the resulting heterozygous F1 was intermediate between that of the completely resistant homozygous parent and the susceptible parent. This result indicates semi-dominant inheritance of the trait of the invention in plants which may comprise the genetic determinant B.

Subsequently these F1 plants were selfed and F2 plants were obtained. In the F2 23% completely resistant plants could be identified, 20% of the plants showed necrotic lesions but no sporulation and 57% of the plants showed symptoms of downy mildew after inoculation. This further confirms the semi-dominant inheritance of genetic determinant A of the invention. The F2 segregation ratio also establishes that the resistance to at least Hyaloperonospora parasitica isolates Pp0615 and Pp1248, is conferred by a monogenetic semi-dominant determinant B.

The invention is further described by the following numbered paragraphs:

1. Rucola plant of the species Diplotaxis tenuifolia comprising genetic determinant B and optionally genetic determinant A that leads to expression of resistance, in particular complete resistance, against at least Hyaloperonospora parasitica isolates Pp0615 and Pp1248, which genetic determinant B is as present in the genome of, or obtainable from, plants grown from seeds of which a representative sample was deposited at the NCIMB under deposit number NCIMB 42151, and which genetic determinant A is as present in the genome of, or obtainable from, plants grown from seeds of which a representative sample was deposited at the NCIMB under deposit number NCIMB 41811.

2. Rucola plant of paragraph 1, obtainable by crossing a first rucola plant with a second rucola plant, wherein one or both of the said plants is grown from seed of which a representative sample was deposited with the NCIMB under deposit number NCIMB 42151, or a progeny plant thereof, and selecting for a plant that shows resistance to at least Hyaloperonospora parasitica isolates Pp0615 and Pp1248, and optionally introgressing genetic determinant A.

3. Rucola plant of paragraph 1 or 2, wherein the plant that comprises a genetic determinant or both genetic determinants is used as the female parent in a cross.

4. Rucola plant of any one of the paragraphs 1-3, wherein genetic determinant B inherits as a single semi-dominant gene and in a homozygous state leads to a complete resistance to at least Hyaloperonospora parasitica isolates Pp0615 and Pp1248 and genetic determinant A inherits as a single dominant gene and leads to a complete resistance to at least Hyaloperonospora parasitica isolates Pp0615 and Pp1248.

5. Rucola plant of paragraph 4, wherein the complete resistance is demonstrated in a bio-assay for Hyaloperonospora parasitica, wherein a relevant number of plants, such as 30, is grown for about 2 weeks at a temperature regime of 14/12° C. day/night before being inoculated with spores of Hyaloperonospora parasitica, in particular spores of Hyaloperonospora parasitica isolates Pp0615 or Pp1248, after which the plants are scored for infection at 10 and 17 days after inoculation, and wherein plants of the invention are characterized by an absence of downy mildew symptoms.

6. Seed of a rucola plant of any of paragraphs 1-5, comprising genetic determinant B and optionally genetic determinant A as defined in paragraph 1.

7. Seed capable of growing into a rucola plant of any of the paragraphs 1-5, comprising genetic determinant B and optionally genetic determinant A as defined in paragraphs 1.

8. Progeny of a rucola plant of any of the paragraphs 1-5, or progeny of a rucola plant grown from rucola seed of paragraph 6 or 7, wherein the progeny of the plant comprises genetic determinant B and optionally genetic determinant A as defined in paragraph 1.

9. Propagation material derived from a rucola plant of any one of the paragraphs 1-5 or from rucola seed of paragraph 6 or 7, wherein the propagation material comprises genetic determinant B and optionally genetic determinant A as defined in paragraph 1.

10. Propagation material capable of growing into a rucola plant of any one of the paragraphs 1-5.

11. Propagation material of paragraph 9 or 10, wherein the propagation material is selected from the group consisting of microspores, pollen, ovaries, ovules, embryos, embryo sacs, egg cells, cuttings, roots, root tips, hypocotyls, cotyledons, stems, leaves, flowers, anthers, seeds, meristematic cells, protoplasts, and cells.

12. Tissue culture of propagation material of any one of the paragraphs 9-11.

13. Plant parts comprising leaves and stems of a rucola plant of any one of the paragraphs 1-5 and 8.

14. Food product comprising plant parts of paragraph 13, in particular leaves and stems, or parts thereof, optionally in processed form.

15. Use of a plant of any one of the paragraphs 1-5 and 8, or plants produced from the seed of paragraph 6 or 7, or from the propagation material of any one of the paragraphs 9-11, as germplasm in a breeding program for the development of a rucola plant that shows resistance against at least Hyaloperonospora parasitica isolates Pp0615 and Pp1248, in particular complete resistance.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A rucola plant of the species Diplotaxis tenuifolia comprising genetic determinant B and optionally genetic determinant A that leads to expression of resistance against Hyaloperonospora parasitica isolates Pp0615 and Pp1248, which genetic determinant B is as present in the genome of, or obtainable from, plants grown from seeds of which a representative sample was deposited at the NCIMB under deposit number NCIMB 42151, and which genetic determinant A is as present in the genome of, or obtainable from, plants grown from seeds of which a representative sample was deposited at the NCIMB under deposit number NCIMB 41811.

2. The rucola plant of claim 1 wherein the expression of resistance is complete resistance, against Hyaloperonospora parasitica isolates Pp0615 and Pp1248.

3. The rucola plant of claim 1, obtainable by crossing a first rucola plant with a second rucola plant, wherein one or both of the said plants is grown from seed of which a representative sample was deposited with the NCIMB under deposit number NCIMB 42151, or a progeny plant thereof, and selecting for a plant that shows resistance to at least Hyaloperonospora parasitica isolates Pp0615 and Pp1248.

4. The rucola plant of claim 3, further comprising introgressing genetic determinant A.

5. The rucola plant of claim 1, wherein the plant that comprises a genetic determinant or both genetic determinants is used as the female parent in a cross.

6. The rucola plant of claim 1, wherein genetic determinant B inherits as a single semi-dominant gene and in a homozygous state leads to a complete resistance to at least Hyaloperonospora parasitica isolates Pp0615 and Pp1248 and genetic determinant A inherits as a single dominant gene and leads to a complete resistance to at least Hyaloperonospora parasitica isolates Pp0615 and Pp1248.

7. The rucola plant of claim 6, wherein the complete resistance is demonstrated in a bio-assay for Hyaloperonospora parasitica, wherein a relevant number of plants is grown for about 2 weeks at a temperature regime of 14/12° C. day/night before being inoculated with spores of Hyaloperonospora parasitica, after which the plants are scored for infection at 10 and 17 days after inoculation, and wherein plants of the invention are characterized by an absence of downy mildew symptoms.

8. The rucola plant of claim 7, wherein the relevant number of plants is 30.

9. The rucola plant of claim 7, wherein the spores of Hyaloperonospora parasitica, are spores of Hyaloperonospora parasitica isolates Pp0615 or Pp1248.

10. A seed of the rucola plant of claim 1, comprising genetic determinant B.

11. The seed of claim 10, further comprising genetic determinant A.

12. A seed capable of growing into a rucola plant of claim 1, comprising genetic determinant B and optionally genetic determinant A.

13. The seed of claim 12, further comprising genetic determinant A.

14. A progeny of a rucola plant of claim 1, wherein the progeny of the plant comprises genetic determinant B.

15. The progeny of claim 14, further comprising genetic determinant A.

16. A progeny of a rucola plant grown from the rucola seed of claim 6, wherein the progeny of the plant comprises genetic determinant.

17. The progeny of claim 16, further comprising genetic determinant A.

18. A propagation material derived from a rucola plant of claim 1, wherein the propagation material comprises genetic determinant B.

19. The propagation material of claim 18, further comprising genetic determinant A.

20. A propagation material capable of growing into a rucola plant of claim 1.

21. The propagation material of claim 18, wherein the propagation material comprises a microspore, pollen, ovary, ovule, embryo, embryo sac, egg cell, cutting, root, root tip, hypocotyl, cotyledon, stem, leaf, flower, anther, seed, meristematic cell, protoplast or cell.

22. A tissue culture of the propagation material of claim 18.

23. The propagation material of claim 20, wherein the propagation material comprises a microspore, pollen, ovary, ovule, embryo, embryo sac, egg cell, cutting, root, root tip, hypocotyl, cotyledon, stem, leaf, flower, anther, seed, meristematic cell, protoplast or cell.

24. A tissue culture of the propagation material of claim 20.

25. A plant part comprising a leaf or a stem of the rucola plant of claim 1.

26. A food product comprising the plant part of claim 25 or a part thereof, optionally in processed form.

27. The food product of claim 26, wherein the plant part is in processed form.

28. A method of developing a rucola plant that shows resistance against Hyaloperonospora parasitica isolates Pp0615 and Pp1248 comprising isolating germplasm from the plant of claim 1 in a breeding program for the development of a rucola plant that shows resistance against Hyaloperonospora parasitica isolates Pp0615 and Pp1248.
